(19) United States
(12) Patent Application Publication
(10) Pub. No.: US 2024/0374912 A1
(43) Pub. Date: Nov. 14, 2024

(54) BioElectric Therapy Program Combined with the Law of Nature, Traditional Chinese Medicine and Electrical Principles

(71) Applicant: Daniel Omar Lopez, San Antonio, TX (US)

(72) Inventor: Daniel Omar Lopez, San Antonio, TX (US)

(21) Appl. No.: 18/395,182

(22) Filed: Dec. 22, 2023

Related U.S. Application Data
(63) Continuation of application No. 63/501,106, filed on May 9, 2023.

(51) Int. Cl. A61N 2/00

(52) U.S. Cl. CPC A61N 2/008

(57) ABSTRACT

BioElectric Therapy is a therapeutic program that utilizes electrical stimulation to promote healing, alleviate pain, and improve overall well-being. This abstract provides an overview of the program, highlighting its principles, techniques, and potential benefits. The efficacy of BioElectric Therapy is supported by scientific research, as well as first-hand experiences and testimonials from clients who have undergone the program. The abstract concludes by emphasizing the potential of BioElectric Therapy as a valuable treatment option for individuals seeking relief from chronic pain, improved mental health, and accelerated healing processes.

### BACKGROUND

A statement of the field of art in which the invention pertains. A paragraph(s) describing to the extent practical the state of the prior art or other information disclosed known to the applicant. Where applicable the problems involved with the art or other information disclosed which are solved by the applicant's invention should be indicated.

The BioElectric Therapy Program is an innovative and alternative approach for pain management and overall wellness with amazing results. This program utilizes bioelectricity energy and electromagnetic energy with various therapeutic techniques to address chronic pain and promote holistic well-being. One of the key features of this program is the use of a Color-coded Health Assessment Report, which provides an easy-to-read and objective measurement of the client's progress and overall health. This report serves as a valuable tool for both healthcare providers and clients, as it correlates the objective data with the subjective feelings and experiences reported by the clients. By combining the objective measurements with the clients' own perceptions, the BioElectric Therapy Program offers a comprehensive and reliable method for monitoring and evaluating the effectiveness of the therapy, ultimately leading to improved pain management and enhanced overall wellness.

### Disclaimer

The BioElectric Therapy program offered by BioElectric Therapy Center is intended to provide energy to the body, enabling the body to function optimally and potentially facilitate the natural healing process. However, it is important to note that this program does not make any medical claims or guarantees regarding the treatment or cure of any specific medical condition.

The BioElectric Therapy program should not be considered a substitute for professional medical advice, diagnosis, or treatment. It is always recommended to consult with a qualified healthcare provider before initiating any new therapeutic program, especially if you have any pre-existing medical conditions or concerns.

While BioElectric Therapy may have positive effects on overall well-being and potentially support the body's self-healing abilities, individual results may vary. The efficacy of this program may depend on various factors, including but not limited to an individual's overall health, lifestyle, and adherence to the program.

### SUMMARY OF THE INVENTION

The summary of the invention, should set out the exact nature, operation, and purpose of the invention, the summary should be directed to the specific invention being claimed in contradistinction to mere generalities of the invention. The subject matter of the invention should be described in one or more clear, concise sentences or paragraphs.

The BioElectric Therapy Program represents a revolutionary approach that combines Traditional Chinese Medicine (TCM) principles with the Law of Nature coupled with basic electronic principles. The combination of ground and voltage provides the fundamental requirements for all electronic devices to work optimally. By harnessing the power of Bioelectricity and aligning it with the body's natural energy flow, this program offers a holistic and comprehensive solution for individuals seeking alternative healthcare options.

In addition to the innovative therapy itself, the BioElectric Therapy Program includes a Health Assessment report. This report serves as an objective tool to monitor the progress of the therapy and provides valuable insights into the client's overall health and well-being. By objectively measuring improvements and tracking changes over time, both healthcare providers and clients can make informed decisions about the effectiveness of the therapy and adjust treatment plans accordingly.

Overall, the BioElectric Therapy Program offers a groundbreaking approach that combines ancient wisdom with modern technology. By integrating TCM, Law of Nature, and electronic principles, this program provides a comprehensive and personalized solution for individuals seeking alternative healthcare options. With the inclusion of a Health Assessment report, clients can objectively monitor their progress and experience the transformative effects of this revolutionary therapy.

### DRAWINGS

### Color Coded Health Assessment Report

The Health Assessment Report contains over 250 parameters. The scan results identify, normal, mildly abnormal, moderately abnormal, severely abnormal/abnormal test results. The color codes are as noted in the color legend of the Health Assessment report. The total number of normal test results determines the percentage score. The report monitors and tracks the percentage score ongoing with BioElectric Therapy sessions. The color code simplifies the interpretation of the BioElectric Therapy Program efficacy with the Health Assessment Report.

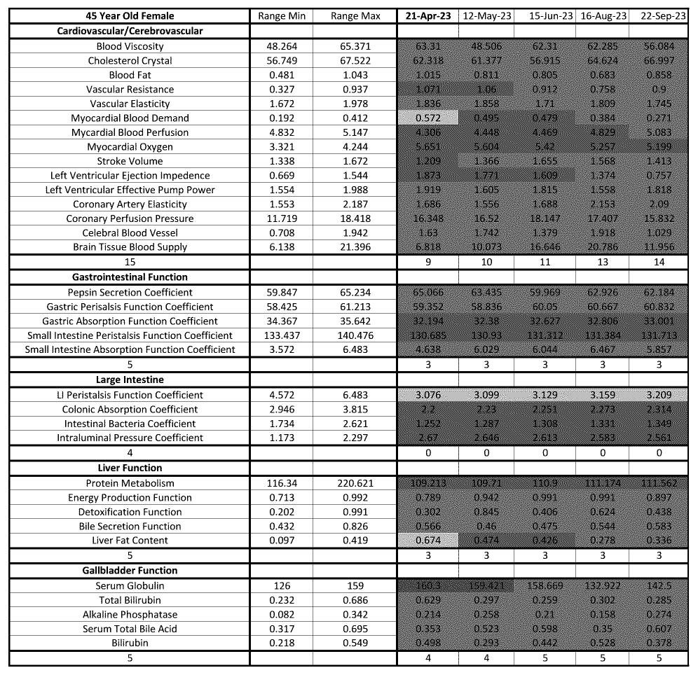

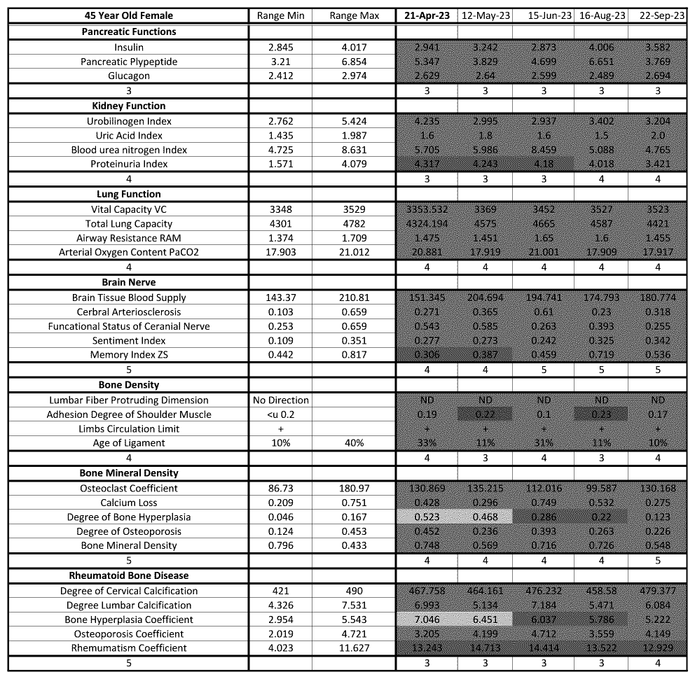

FIG. 1a-h: Health Assessment report of 23-Year-Old Female. Normal test results Score from 55% to 82% in 22 months.

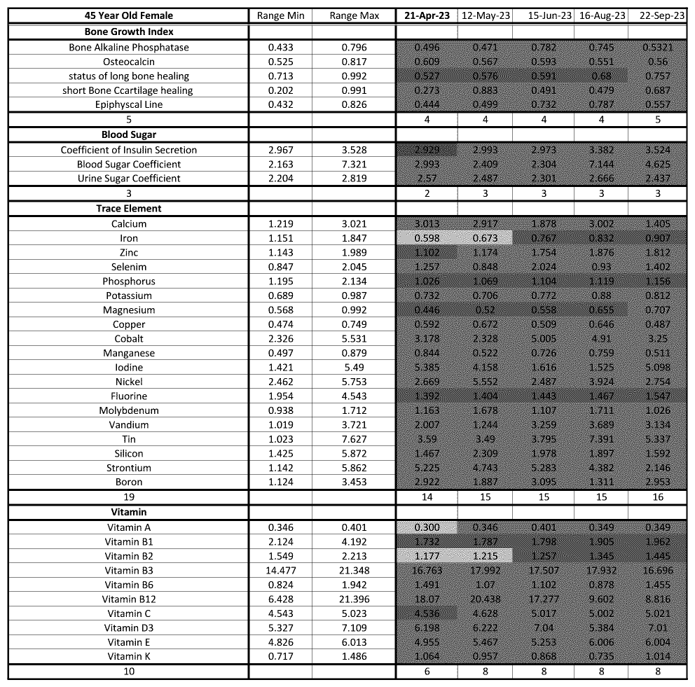

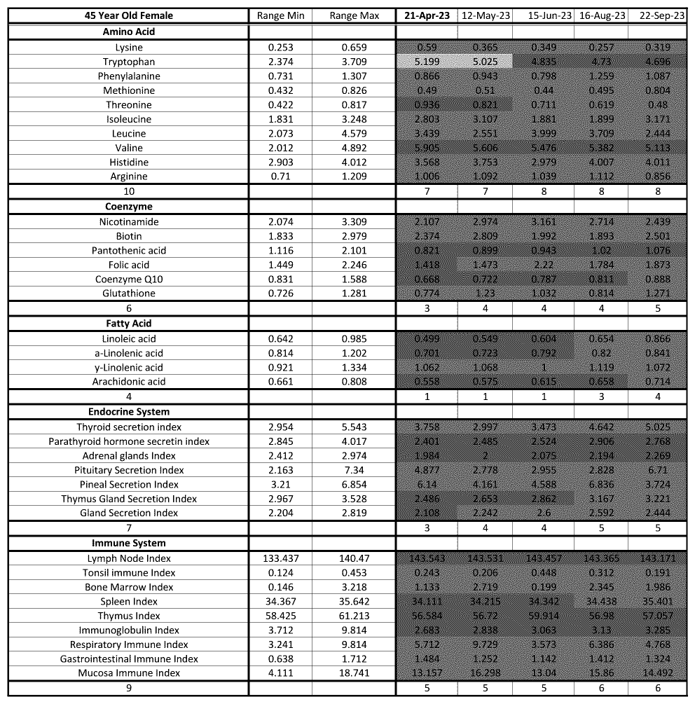

FIG. 2a-h: Health Assessment Report of 66-Year-Old Female. Normal Test Results Score from 49% to 79% in 16 months

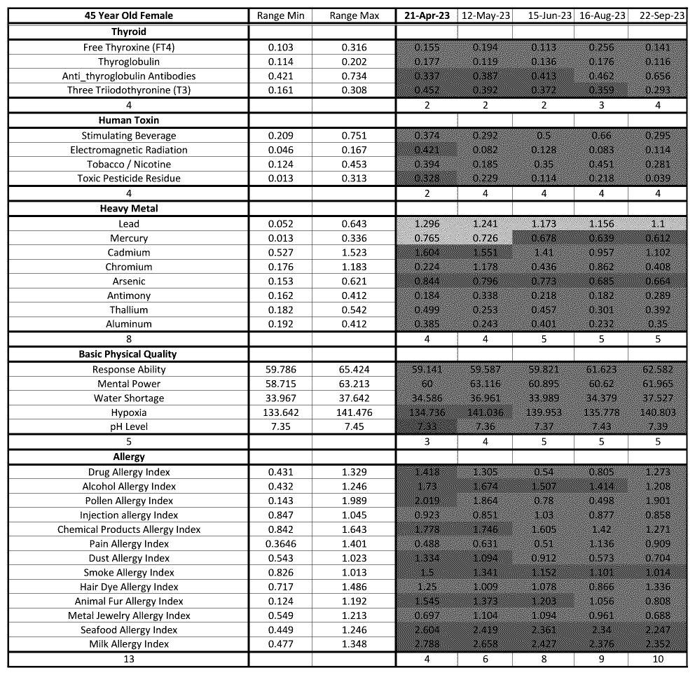

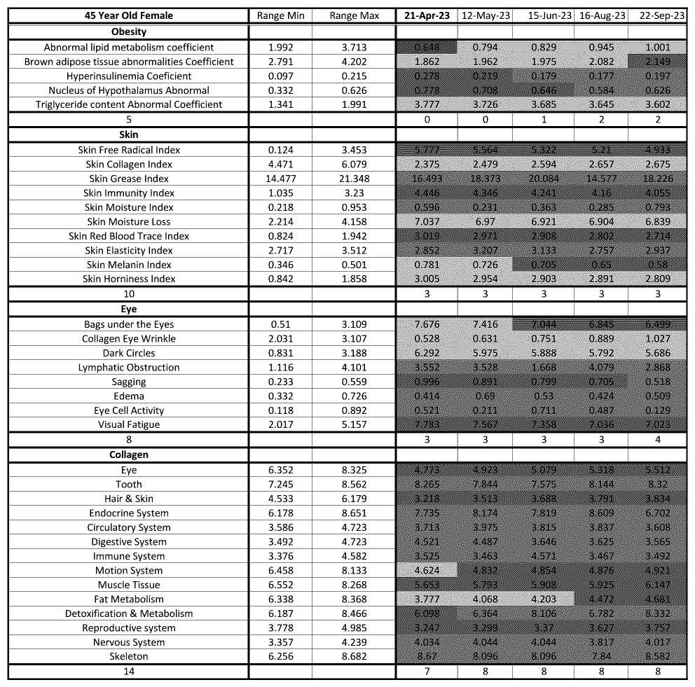

FIG. 3a-h: Health Assessment Report of 77-Year-Old Female. Normal Test Results Score from 52% to 77% in 4 months

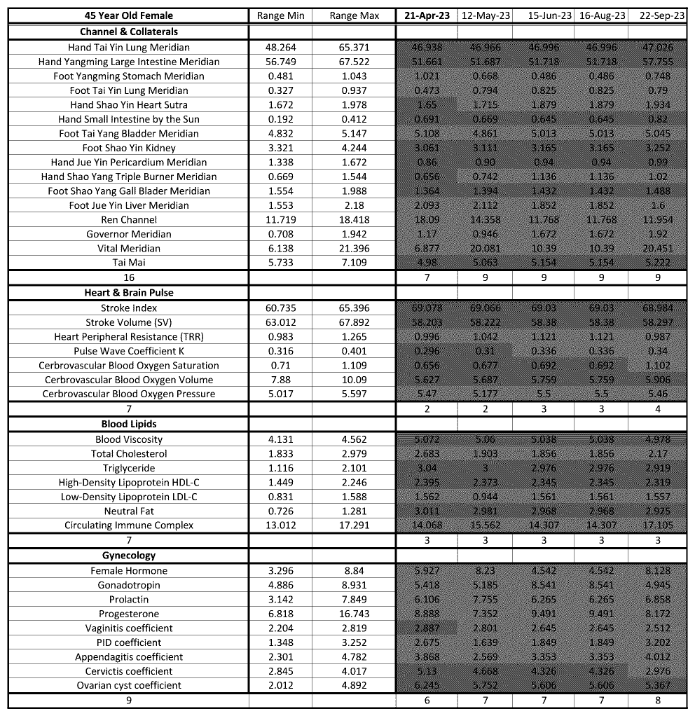

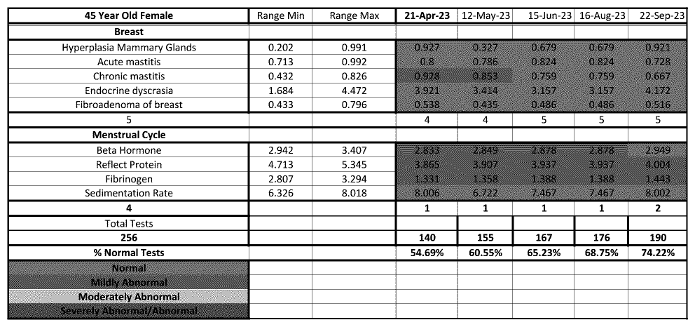

FIG. 4a-h: Health Assessment Report of 12-Year-Old Male. Normal Test Results Score from 55% to 77% in 4 months.

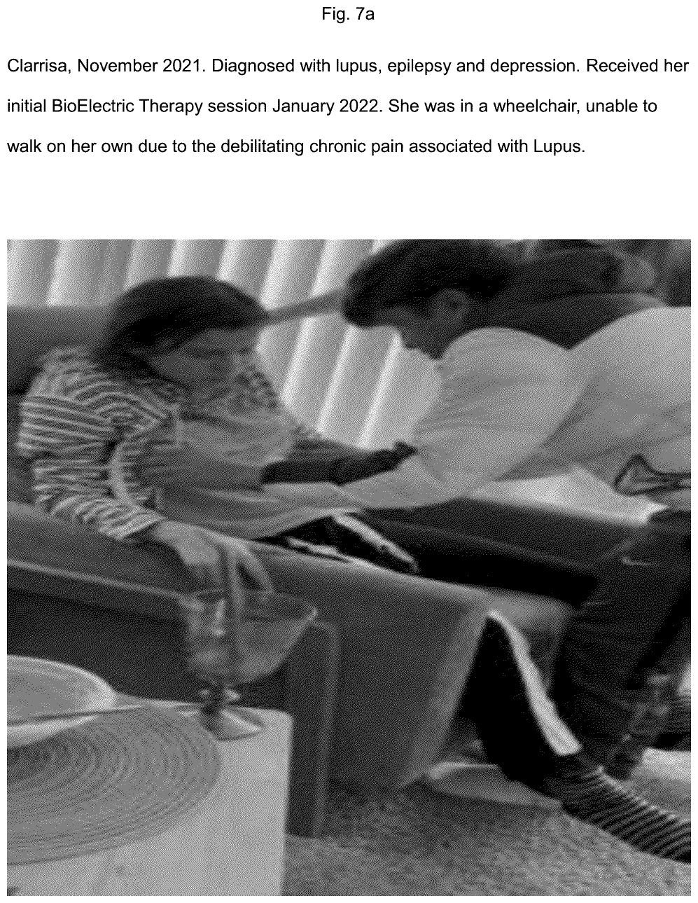

FIG. 5a-h: Health Assessment Report of 80-Year-Old Male. Normal Test Results Score from 52% to 70% in 4 months.

FIG. 6a-h: Health Assessment Report of 45-Year-Old Female. Normal Test Results Score from 54% to 74% in 5 months.

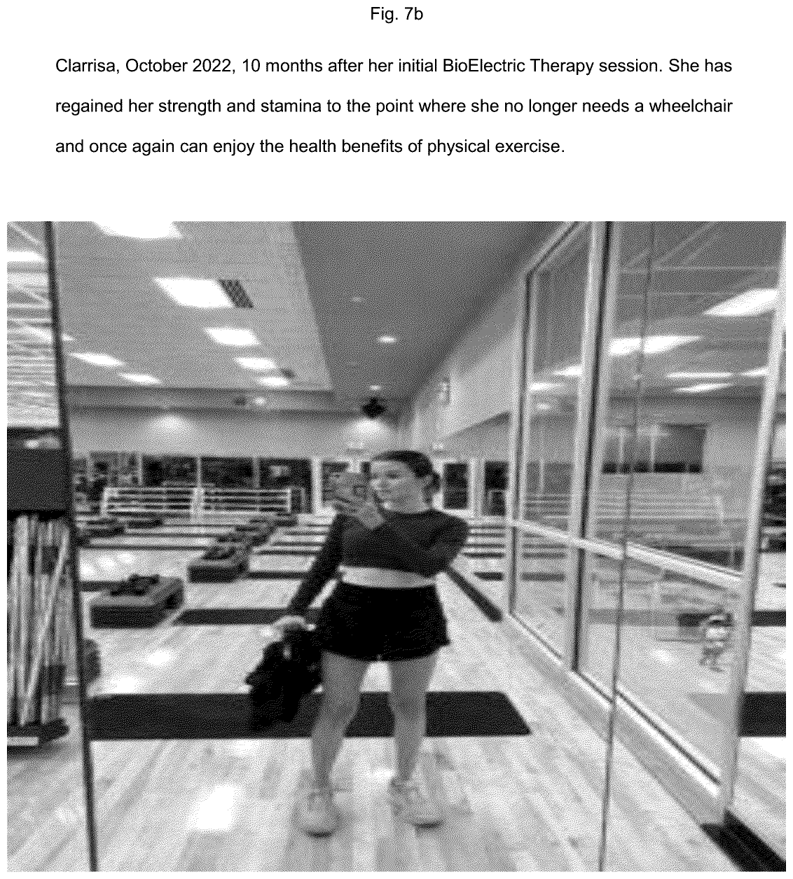

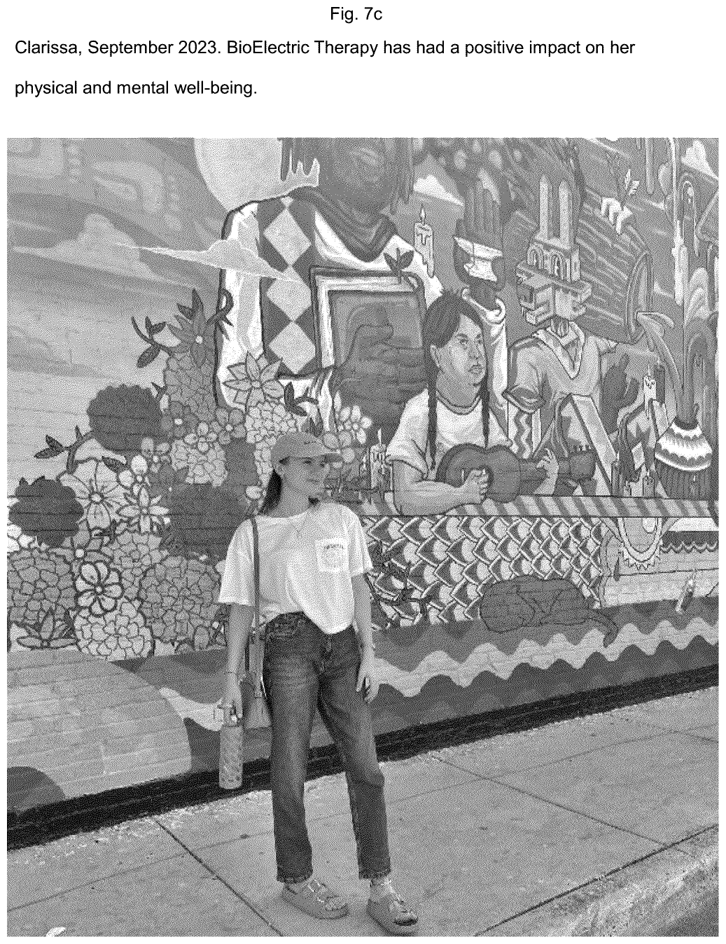

FIG. 7a-c: Before and After Photos of 22-Year-Old Female diagnosed with Lupus, Epilepsy and Depression.

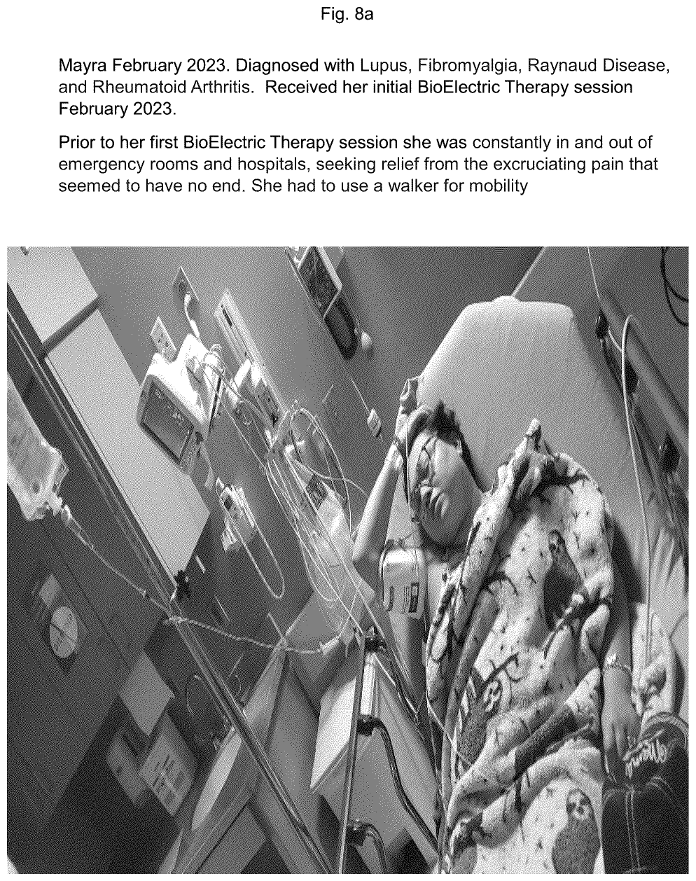

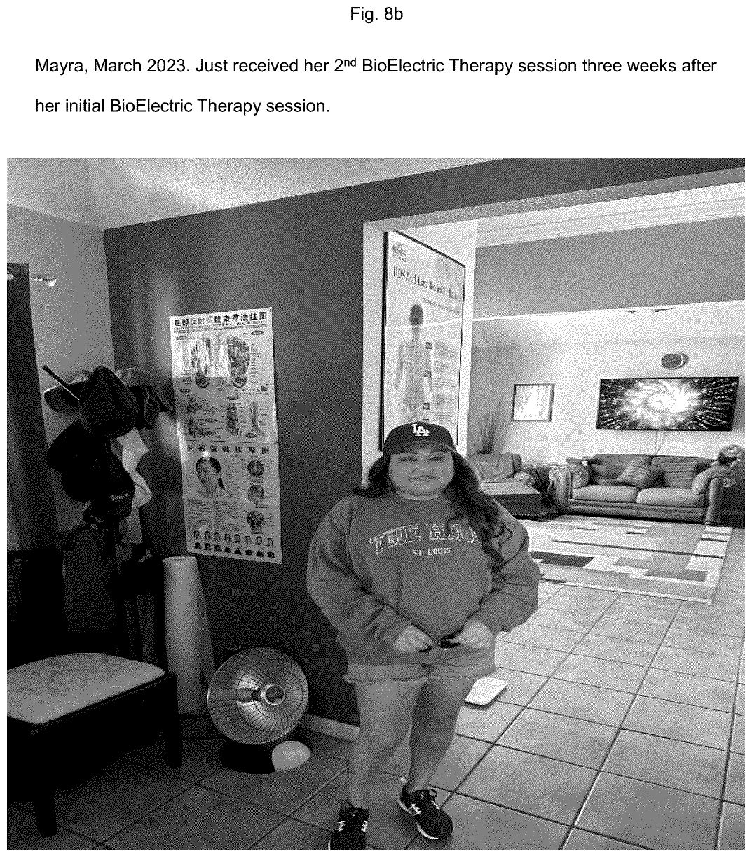

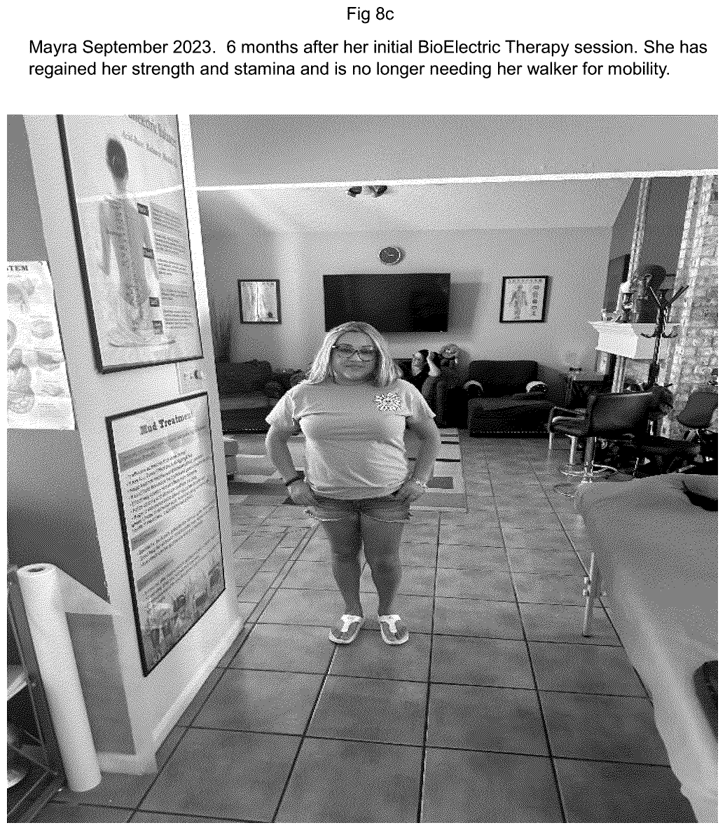

FIG. 8a-c: Before and After Photos of 46-Year-Old Female diagnosed with Lupus, Fibromyalgia, Raynaud disease, and Rheumatoid Arthritis.

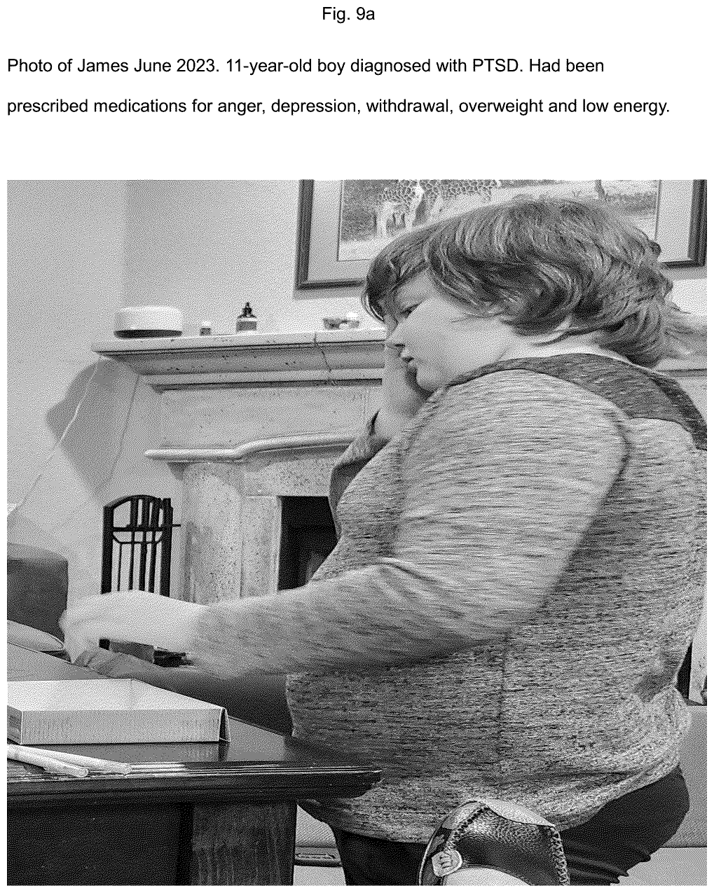

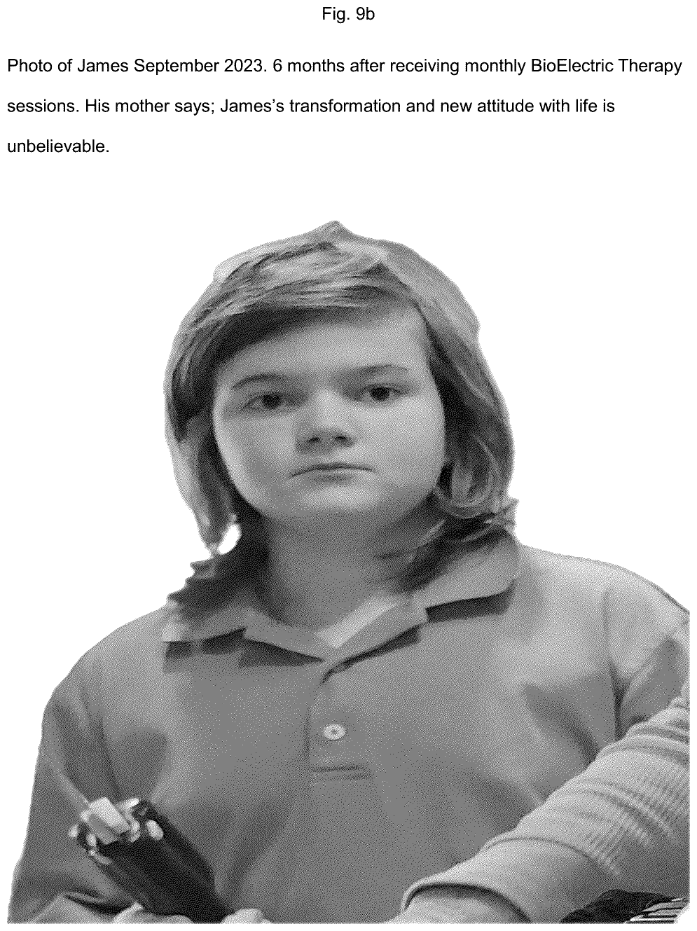

FIG. 9a-b: Before and After Photos of 12-Year-Old Male diagnosed with PTSD, panic attacks extreme restlessness, depression, low energy, overweight and overall emotional unbalance.

### DETAILED DESCRIPTION

The detailed description must be in such particularity as to enable any person skilled in the pertinent art or science to make and use the invention without extensive experimentation. Applicant is permitted to use this or her own terminology, as long as it can be understood. The subject matter of the invention should be described in one or more clear, concise sentences or paragraphs.

The BioElectric Therapy program is founded on the fundamental principles of Traditional Chinese Medicine (TCM) and the law of nature. By integrating these ancient wisdoms with modern technology, this program offers a holistic and comprehensive approach to wellness and healing. One of the key components of this program is the recognition of the two basic requirements for all electronic devices to operate optimally: grounding and proper voltage.

Grounding is essential for maintaining the balance and flow of energy within the body. In the BioElectric Therapy program, grounding techniques are incorporated to help individuals connect with the earth's natural energy and restore harmony within their own energy systems. This grounding aspect not only promotes physical well-being but also supports emotional and mental balance.

Proper voltage, on the other hand, ensures the efficient functioning of electronic devices. In the context of the BioElectric Therapy program, this principle is applied to optimize the therapeutic effects of the therapy. By aligning the body's bioelectricity with the appropriate voltage, the program aims to enhance the body's natural healing mechanisms and promote overall wellness.

By combining the principles of TCM and the law of nature with the understanding of grounding and proper voltage, the BioElectric Therapy program offers a unique and effective approach to wellness and healing. This program recognizes the importance of harmonizing the body's energy and optimizing its functioning, ultimately leading to improved health, pain management, and overall well-being.

Just like electronic devices, humans also require proper grounding and voltage levels to operate optimally. Without adequate grounding, we may feel disconnected, unbalanced, and unable to effectively manage our energy. This can lead to physical, emotional, and mental imbalances, ultimately impacting our overall well-being.

Similarly, having the right voltage is crucial for humans to function at their best. When our energy levels are low or inconsistent, we may experience fatigue, lack of focus, and decreased productivity. Over time, this can lead to burnout and breakdown, just like an electronic device that fails due to inadequate voltage.

Recognizing the importance of grounding and proper voltage, the BioElectric Therapy program aims to restore and optimize these essential elements within the human body. By aligning our energy with the earth's natural grounding and ensuring a harmonious flow of bioelectricity, this program promotes optimal functioning, pain management, and overall wellness.

Just as electronic devices require grounding and proper voltage to operate optimally, humans also need these elements to function at their best. The BioElectric Therapy program acknowledges this fundamental principle and offers a holistic approach to restore and optimize grounding and voltage within the human body, ultimately supporting optimal health, well-being, and preventing breakdown.

In Traditional Chinese Medicine (TCM), meridians are considered the channels through which energy, known as “qi,” flows in the body. These meridians facilitate the communication and exchange of energy, transmitting vital information throughout the body. The BioElectric Therapy program recognizes the significance of these meridians and their role in maintaining health and well-being.

BioElectric Therapy works by opening up these pathways through the application of voltage to the meridians and acupuncture points. By doing so, the therapy enhances the flow of bioelectric energy, or “qi,” throughout the body. This increased flow of energy provides the body with the vital energy it needs to heal itself and restore balance.

By stimulating the meridians and promoting the flow of bioelectric energy, the BioElectric Therapy program aligns with the principles of TCM. It recognizes that when the pathways are open and the energy is flowing freely, the body can access its innate healing abilities and achieve optimal health.

The Law of Nature/Physics emphasizes the crucial role of maintaining a balanced pH level in our bodies. This balance is essential as it provides the necessary voltage for cells to regenerate as healthy cells. When the voltage is inadequate, cells may regenerate as sub-healthy or sick cells, which can contribute to the development of chronic illnesses.

It is interesting to note that as a society, we often prioritize monitoring the pH level of water in our swimming pools rather than in our own bodies. However, it is imperative that we recognize the significance of maintaining a proper voltage by ensuring a pH level within the range of 7.35 to 7.45.

By actively maintaining this balanced pH level, we can support the optimal regeneration of healthy cells and promote overall well-being. This understanding aligns with the principles of the Law of Nature/Physics, emphasizing the importance of maintaining a balanced pH level to ensure the proper voltage required for cellular regeneration.

The Law of Nature/Physics highlights the significance of maintaining a balanced PH level in our bodies. This balance is crucial for providing the appropriate voltage necessary for cells to regenerate as healthy cells. By prioritizing and actively maintaining a pH level within the range of 7.35 to 7.45, we can support the regeneration of healthy cells and contribute to our overall health and wellness.

The pH level determines the appropriate voltage, which is crucial for human well-being. It is essential for humans to lean towards the alkaline side rather than the acidic side. To ensure healthy cell regeneration, the pH level should fall within the range of 7.35 to 7.45, providing the necessary −20 to −25 millivolts. When our acidity increases, the voltage available to cells decreases.

To illustrate the difference between acidic and alkaline conditions, imagine a fish bowl. In an acidic state, the water is dirty and murky, the fish appear sluggish, sickly, and lack energy. On the other hand, in an alkaline environment, the water is clean and pristine the fish are lively, active, and full of energy.

Regrettably, due to the Standard American Diet (SAD), environmental factors, and stress, the majority of people find themselves in an acidic state. This acidic condition sets off a negative chain of events, leading to inflammation and a multitude of chronic illnesses and diseases.

The BioElectric Therapy program represents revolutionary approach to pain management and overall health and wellness. This program consists of three essential components.
1. Pulse Electromagnetic Field/PEMF
2. Acid Base BioElectric Direct Detoxification System/DDS
3. Quantum Resonance Magnetic Analyzer/QRMA

The principle of yin and yang is rooted in the concept of balance. In the BioElectric Therapy program, this balance is achieved through two fundamental components: Earthing/Grounding represents the yin aspect, while BioElectric energy embodies the yang aspect. Both elements are essential and necessary for the successful implementation of the BioElectric Therapy program.

### Pulse Electromagnetic Field/PEMF

The initial component of the BioElectric Therapy program is the Pulse Electromagnetic Frequency (PEMF), which serves as the yin aspect of the therapy. The PEMF is responsible for delivering the grounding/earthing electromagnetic energy. The electromagnetic energy emitted by the PEMF acts as a comprehensive body recharger. Its electromagnetic fields effortlessly penetrate all tissues and cells, facilitating profound healing. This process enhances your energy levels and vitality, providing similar benefits to those experienced through grounding/earthing.

### Acid Base BioElectric Direct Detoxification System/DDS

The second component of the BioElectric Therapy program is the Acid Base BioElectric Direct Detoxification System (DDS). This component represents the yang aspect of the therapy, supplying the necessary Qi/BioElectric energy to charge and sustain the appropriate voltage in our cells and bodies. This process is akin to the electrical charge that the earth receives through lightning strikes occurring worldwide. It is comparable to the feeling of cleanliness and revitalization experienced after a rainstorm, when the earth becomes charged.

Similar to acupuncture, the BioElectric Therapy program utilizes the concept of pulling electrical energy from the environment. However, instead of using needles to open up and unblock the meridian channels and acupuncture points, the BioElectric Therapist applies BioElectric energy through their hands. This energy is directed towards the blocked meridians and acupuncture points, serving to remove the blockages. Just like a kinked hose restricts the flow of water, blockages in the meridians hinder the proper flow of Qi or energy. By clearing these blockages, the Qi can flow smoothly through the meridians. Consequently, improved circulation of blood and enhanced oxygen supply to the body organs are achieved, allowing for optimal bodily function.

### Quantum Resonance Magnetic Analyzer/QRMA

The third component of the BioElectric Therapy program is the Quantum Resonance Magnetic Analyzer (QRMA). This biofeedback whole body scanner operates based on the principles of electromagnetism. It detects and analyzes the electromagnetic wave signals emitted by the human body, which reflect the specific state of the body. These electromagnetic wave signals vary depending on whether the body is in a healthy, sub-healthy, or illness state.

The QRMA device gathers the subtle magnetic fields emitted by human cells to conduct a scientific analysis. It compares and examines each organ, enabling the determination of a person's overall health. This analysis is non-invasive in nature. The Health Assessment Report generated by the QRMA provides comprehensive test results for men (248) and women (256), covering various aspects from head to toe. Throughout the BioElectric Therapy program, the effectiveness of the therapy is continuously tracked and monitored. The QRMA device stands out as a unique and cutting-edge tool for human bioenergetic and biochemical analysis, offering valuable insights into the health or illness state of the client. The device collects the electromagnetic resonance signals emitted by the human body, which reflect the various health conditions such as healthy, sub-health, and illness. These signals provide valuable information about the overall health condition of the individual.

The Health Assessment report generated by the BioElectric Therapy Program is designed to be user-friendly and visually intuitive, utilizing color-coded indicators. This report serves as a valuable tool for both the client and the therapist to track and monitor the progress and effectiveness of the BioElectric Therapy. It stands out as one of the few natural alternative therapies that offer an objective and ongoing health assessment report, providing valuable insights into the client's health journey

### Actual Cases

In order to address the question of whether BioElectric Therapy really works, I would like to present a range of first-hand experiences and testimonials from clients who have undergone this therapy program. These accounts provide compelling evidence of the efficacy and remarkable results achieved through this BioElectric Therapy program.

These first-hand experiences and testimonials provide compelling evidence of the efficacy of BioElectric Therapy. They demonstrate its ability to alleviate chronic pain, improve mental health conditions, and accelerate healing processes. While individual results may vary, these accounts collectively support the notion that BioElectric Therapy program can indeed deliver remarkable and life-changing outcomes for clients.

These remarkable results and positive testimonials from clients who have undergone BioElectric Therapy provide strong support for its efficacy. While further research and studies may be necessary to fully understand the mechanisms behind BioElectric Therapy, the evidence presented here suggests that it is a valuable and effective treatment option for those seeking relief and improved well-being.
1. 23 Y/O Female diagnosed with Lupus and Epilepsy contacted me and wanted to know if BioElectric Therapy could help her with her illnesses. In addition to Lupus and Epilepsy, she was very depressed, weak, had insomnia and was in a wheel chair. After the first session, I could see it in her eyes that the BioElectric therapy had helped her. She became more alert and had more energy. She is feeling much better, stronger, simply amazing. She has regained her life, continuing her education, playing soccer, rock climbing and other physical sports.

Testimony: I am absolutely thrilled to share my life-changing experience with BioElectric Therapy and the incredible impact it has had on my health and overall well-being. Prior to discovering this revolutionary therapy, I was diagnosed with lupus and epilepsy, which left me feeling utterly defeated and trapped in a cycle of pain, fatigue, and depression. I was confined to a wheelchair, unable to enjoy the simplest pleasures of life.

However, everything changed when I decided to give BioElectric Therapy a try. From the very first session, I noticed a significant reduction in pain and an increase in energy levels. The debilitating fatigue that had plagued me began to dissipate, allowing me to regain control over my life. I could finally envision a future where I could walk freely and engage in activities that I had long given up on.

Not only did BioElectric Therapy alleviate my physical symptoms, but it also had a profound impact on my mental health. The depression that had consumed me began to lift, and I found myself feeling more optimistic and hopeful about the future. The therapy provided me with a renewed sense of purpose and motivation to overcome the challenges I faced.

As I continued with BioElectric Therapy, I regained my strength and mobility. The wheelchair that had become my constant companion was no longer necessary, and I could walk independently once again. The freedom and joy I experienced from being able to move without pain were indescribable. It was as if a weight had been lifted off my shoulders, and I could finally embrace life to its fullest.

I am eternally grateful for the transformative power of BioElectric Therapy. It has given me a second chance at life, allowing me to break free from the confines of my illnesses and reclaim my independence. I now live a life filled with vitality, joy, and purpose. If you are struggling with chronic pain, fatigue, or any other health challenges, I wholeheartedly recommend BioElectric Therapy. It has the potential to change your life in ways you never thought possible.
2. 46 Y/O Female with Ovarian Cyst. Qi/BioElectric energy applied to specific acupuncture points along with the standard BioElectric Therapy procedure. She had 2 sessions, but after the 2^{nd} session, the client reported pain and excessive bleeding. The following day, she saw her OBGYN, and informed her, the cyst had ruptured. The body was healing itself and she would no longer need the surgery.

Testimony: I have had 2 experiences with Dan Lopez and BioElectric Therapy and although we are not to make medical claims, I recommend without a doubt investigating this therapy before consulting modern medicine. First situation I had a knot on my foot from a trauma that left me almost unable to even walk with it. Two months I struggled with no relief. One session with BioElectric Therapy and not only did I land on that foot when I got off the table and walked pain free, three days later the knot was completely gone. The second situation I strongly urge women to pay attention with a 4 cm ovarian cyst the gyn just wanted to cut, cut out that ovary and my uterus because of my age 46? I am not ovulating for whatever reason, but my hormone levels are perfect and the biopsy of the uterus is normal. BUT LET'S CUT OUT THE OVARY AND UTERUS and cause what other problems? Two sessions with BioElectric Therapy and it ruptured. The gyn said; “I have read online that cysts do sometimes burst on their own”. This one was there a long time I JUST KNOW it was the BioElectric Therapy and Acupuncture Points of treatment that saved me a lot of money and future hormone and health issues; you must ask yourself why are they so quick to push you to: cut it out.
3. 65 Y/O Male diagnosed with Fatigue, Insomnia and Post Traumatic Stress Disorder. The following morning after his initial session, he was pleasantly surprised with the amount of energy he felt and a certain calmness that he had not felt in quite some time. Since then, he has continued regular treatment and has reported sleep improvement and calmness. His PTSD symptoms have reduced and medication has been reduced from 3 pills a day to 1 pill a day for PTSD and only if necessary.

Testimony: I can't say enough about my experience with Dan Lopez and BioElectric Therapy—the past ten years when I retired from the U.S. Marine Corps. Like many veterans, I was diagnosed with PTSD, pending left knee replacement, sleep apnea, and feeling tired most of the time. I was on several different types of medication over the years. Massage therapy was relaxing, but I continued to have a great deal of tension, and my muscles were always tight. I was irritable and anxious most of my days. My massage therapist referred me to Dan to try BioElectric Therapy. Since my first visit, I have been his client for two years. BioElectric Therapy reduces my muscle tension and stress. The pain in my knee is not as bad, and I am sleeping much better. My meds have also been reduced. I feel better and stronger. Dan is very knowledgeable and meticulous in this field. He stays current on the technology and techniques to serve his client better. I highly recommend this treatment BioElectric Therapy for those with chronic pain.
4. 69 Y/O Male with no serious acute or chronic issues but with a belief in Natural Medicines was recommended by his daughter to give BioElectric Therapy a try. He was very curious and anxious try to this new approach for healthcare to see what could possibly be the outcome.

Testimony: I've received two bio-electric treatments a month from Dan Lopez and BioElectric Therapy for a little over a year. When I started, I had no health problems, so my testimonial isn't going be as dramatic as most. I'm 69 years old and very interested in slowing down the aging process and I feel that these treatments are doing that. My overall health score, as measured by the Health Assessment Report, was 55% before starting any BioElectric Therapy treatments and has gradually risen up to 78%. I consider that to be wonderful progress. The biggest improvements have been to my prostate which has changed from slightly enlarged to normal, my testosterone level, my memory, my osteocalcin level, the increase of the level of collagen in my bones, and the removal of toxic mercury from my body. Another, very interesting point is that several vitamins and minerals that I was low in were identified by the Health Assessment Report. I then took the needed supplements and the tests showed very good improvements.
5. 42 Y/O Female diagnosed with a diagnosis of Lupus since 2019. I was very excited and anxious to see if I could replicate the results with a second Lupus client with the BioElectric Therapy program. I believed, it I could get similar results as with my first Lupus client, this therapy could be used as a therapy to help many others with the debilitating chronic condition to improve their quality of life with Lupus. She had to quit working and was using a walker to be mobile. She had just been released from the hospital 2 days prior to her initial BioElectric Therapy session. She came in using a walker to and needed assistance to get on the table. After the initial session, to my surprise, she returned 3 weeks without the aid of her walker and had gone back to work. She has now been able to improve her quality of life with a new approach for managing and living with a Lupus diagnosis. Her doctors are saying, “whatever you are doing, keep doing it”.

Testimony: I cannot express enough how grateful I am for BioElectric Therapy and the incredible impact it has had on my life. For the past 14 years, I have been battling Lupus, fibromyalgia, Raynaud disease, and rheumatoid arthritis, which have caused me immense pain and suffering. The past three years have been particularly challenging, as I found myself constantly in and out of emergency rooms and hospitals, seeking relief from the excruciating pain that seemed to have no end.

I had reached a point where I had to rely on a walker just to move around, and even that was a struggle. The pain had taken away my ability to enjoy the simplest pleasures in life, such as playing with my beloved grandchildren. It felt as though my world was crumbling around me, and I had lost all hope of ever finding relief.

That was until I discovered BioElectric Therapy. Skeptical at first, I decided to give it a try, desperate for any glimmer of hope. After my very first session, I could hardly believe the results. Despite the four-hour return trip home, I was able to walk slowly without my walker. It was a small victory, but it filled me with a renewed sense of hope and determination.

Encouraged by this initial success, I decided to commit to regular monthly treatments. And let me tell you, it has been nothing short of life-changing. With each session, I have experienced a gradual reduction in pain and an increase in mobility. The constant visits to the ER and hospitals have become a thing of the past, and I am finally able to live a more normal life.

Thanks to BioElectric Therapy, I have been able to return to work, something I never thought would be possible again. The joy of being able to play with my grandchildren, to run around and laugh with them, is indescribable. It's as if I have been given a second chance at life, and I am determined to make the most of it.

I cannot thank BioElectric Therapy Center enough for developing such a groundbreaking treatment. Their dedication and expertise have given me back my independence, my joy, and my ability to live life to the fullest. If you are someone who, like me, has been suffering from chronic pain with no relief in sight, I urge you to give BioElectric Therapy a chance. It may just be the miracle you've been waiting for.
6. 65 Y/O Female with Sit-Bone Pain unable to sit without pain. She felt some relief after the first session, but after the second session, she no longer felt the chronic sit bone pain and was very encouraged with the relief she feels and to her surprise with her digestive system. Her digestive system was inflamed and had undergone with a digestive surgery procedure removing some of her colon over 3years ago. She was not able to eat without experiencing digestive pain and would rely on Tums or Pepto Bismol for some digestive pain relief. After 2months of Bioelectric Therapy session, she is now able to eat without any pain with her digestive system and she is also able to sit comfortably without any sit-bone pain, while she is eating.

Testimony: Approximately 7 years ago, I started experiencing acute pain radiating from my sit bone, down my leg, to my foot. I was prescribed pain medication and physical therapy. I briefly took the pain meds but decided to focus on the physical therapy, which worked for a while until it didn't. My physician recommended more pain meds and additional physical therapy. Just when I accepted this pain as my new norm, I heard about the BioElectric Therapy Center. Based on the positive reviews and testimonies, I decided to give it a try. I am happy to say, I have been a client for 2 months now. The treatments are enjoyable, extremely relaxing, and I am now referred to as the Eveready Bunny because my energy level is off the charts! My sits bone pain is no more, and I couldn't be happier! To add to my positive experience, I gained a SURPRISE bonus-I no longer am battling the horrible symptoms of IBS and Diverticulitis of which I have had for 35 years. My gastroenterologist suggested multiple diets to work around my symptoms, but none have worked. I am now able to eat basically anything without the unwelcomed symptoms I had been accustomed to.
7. 67 Y/O Female with chronic fatigue, lack of energy, weight gain, and insomnia. Thinking all of these symptoms are a part of aging, but as a Reiki practitioner she knew the benefit of moving energy within the body and wanted to experience BioElectric Therapy.

Testimony: My experience with Bioelectric therapy has been very positive for me. At first, I did not know what it was like and what it did in our bodies. I have had BioElectric Therapy every month for over a year and I am very happy. At first, I wanted it to help me with asthma and to improve the hearing of one of my ears that once used a hearing aid. Now, I don't need to take medicine for my asthma and my hearing has improved noticeably. On a scale of 1 to 10, it was 3to 5 sometimes and now it's 9.5. This therapy not only helped me with this but with other aspects as well, feeling and sleeping well and much more. It is like a massage with vibration unblocking what's blocked in the body and does not flow as it should. It is not invasive which I prefer.
8. 67 Y/O Female with chronic fatigue, body aches, insomnia, irritable, lack of mental clarity and poor bowel movements was recommended by her massage therapist to try BioElectric Therapy. She complained life was more difficult due to her many ailments she has been going through for several years but was open to try an alternative method of healthcare. She was so pleased with the results from her first she has brought her husband for BioElectric Therapy.

Testimony: I am thrilled to share my incredible experience with BioElectric Therapy and the remarkable benefits it has brought into my life. Before discovering this revolutionary therapy, I was constantly plagued by chronic pain that made it difficult for me to walk and carry out my daily activities.

Since incorporating BioElectric Therapy into my routine, I have witnessed a significant reduction in pain, allowing me to walk without any discomfort. One of the most remarkable changes I have noticed is the improvement in my bowel movements. Previously, I struggled with irregularity and digestive issues, which often left me feeling bloated and uncomfortable. However, after undergoing BioElectric Therapy sessions, my bowel movements have become more regular and efficient, providing me with a newfound sense of relief and comfort.

BioElectric Therapy has had a profound impact on my sleep patterns. Prior to this therapy, I would often toss and turn throughout the night, struggling to find a restful sleep. I have experienced a remarkable improvement in the quality of my sleep. I now wake up feeling refreshed and rejuvenated, ready to take on the day ahead.

Additionally, the physical benefits, BioElectric Therapy has also had a positive impact on my mental well-being. I have noticed a significant increase in mental clarity, allowing me to think more clearly and make decisions with ease.

Furthermore, I have become less irritable and more relaxed, which has greatly improved my overall mood and interactions with others. Overall, BioElectric Therapy has been a life-changing experience for me. It has alleviated my chronic pain, improved my bowel movements, enhanced the quality of my sleep, and provided me with mental clarity and emotional stability. I am incredibly grateful for this therapy and would highly recommend it to anyone seeking a natural and effective solution to their health concerns.
9. 12 Y/O Male with PTSD, panic attacks extreme restlessness, depression, low energy, overweight and overall emotional unbalance. On his initial visit, he was angry and withdrawn, but after his first BioElectric Therapy session, his attitude and disposition changed for the better.

Testimony: I am beyond grateful to have discovered the life-changing benefits of BioElectric Therapy for my 12-year-old son. Words cannot adequately express the profound impact this therapy has had on his overall well-being and quality of life. From the moment we began this journey, it became abundantly clear that BioElectric Therapy was the missing piece to his puzzle of healing.

Prior to starting BioElectric Therapy, my son was plagued by a myriad of challenges that seemed insurmountable. Anxiety and panic attacks had become a daily occurrence, leaving him paralyzed with fear and unable to fully engage in life. His restlessness was so extreme that it hindered his ability to focus and concentrate, affecting his academic performance and social interactions. The weight of depression weighed heavily on his young shoulders, robbing him of the joy and enthusiasm that should accompany childhood. It seemed as though he was trapped in a constant state of emotional turmoil, desperately seeking relief.

However, everything changed when we discovered BioElectric Therapy. From the very first session, we witnessed remarkable results that surpassed our wildest expectations. The therapists at BioElectric Therapy provided a safe and nurturing environment, allowing my son to express his fears and anxieties without judgment. Their expertise and compassion guided him through the healing process, empowering him to regain control over his emotions and find inner peace.

The transformation we witnessed in our son was nothing short of extraordinary. His anxiety and panic attacks diminished significantly, allowing him to navigate life's challenges with newfound confidence and resilience. The restlessness that once consumed him gradually subsided, enabling him to focus and concentrate on his studies and social interactions. The weight of depression lifted, revealing a bright and joyful spirit that had been buried beneath the surface. BioElectric Therapy provided him with the tools to navigate his emotions, fostering self-awareness and emotional regulation that he had previously struggled to attain.

In addition to addressing his emotional well-being, BioElectric Therapy also alleviated physical discomforts that had plagued him. Growing pains, which had previously disrupted his sleep and caused discomfort, became more manageable and less frequent. His energy levels increased, allowing him to engage in activities with enthusiasm and vigor. Furthermore, BioElectric Therapy played a crucial role in weight management, helping him achieve a healthier and more balanced lifestyle.

Today, my son stands as a testament to the incredible power of BioElectric Therapy. He is no longer held captive by anxiety, restlessness, depression, or low energy. Instead, he exudes a sense of calm, confidence, and emotional balance that is truly awe-inspiring. BioElectric Therapy has given him the tools to navigate life's challenges with grace and resilience, setting him on a path towards a bright and promising future.

To any parent who may be grappling with similar challenges, I wholeheartedly recommend BioElectric Therapy as a beacon of hope. The transformative effects it has had on my son's life are nothing short of miraculous. Embrace this opportunity for healing and watch as your child blossoms into their true potential. BioElectric Therapy is a gift that keeps on giving, and I am eternally grateful for the positive impact it has had on my son's journey towards emotional and physical well-being.
10. 45 Y/O Female with low energy and mental exhaustion and neck pain from the stress of life and other life events. Looking for alternative treatment for overall wellness and more energy.

Testimony: What has Bioelectric Therapy done for me? I am overwhelmed with gratitude as I reflect upon the incredible impact that BioElectric Therapy has had on my physical, emotional, and spiritual well-being. Prior to discovering this remarkable therapy, I was burdened by a multitude of health challenges that seemed insurmountable. From anxiety and neck pain caused by TMJ to back pain resulting from a previous back injury, arthritis that affected my entire body, and an unidentified inflammatory condition, my quality of life was greatly diminished. Additionally, I battled with anxiety, depression, weight management issues, and struggled to regulate my emotions. However, BioElectric Therapy has been a true game-changer in my healing journey.

From the very first session, I felt a sense of hope and relief wash over me. The compassionate and knowledgeable therapists at BioElectric Therapy took the time to understand my unique challenges and developed a personalized treatment plan that addressed my specific needs. Their expertise and support created a safe space for me to open up about my struggles and fears, allowing for a truly transformative healing experience.

The results I have experienced have been nothing short of miraculous. The anxiety that once consumed me has significantly diminished, allowing me to navigate life's challenges with newfound calm and resilience. The neck pain caused by TMJ has greatly improved, providing me with much-needed relief and restoring my ability to enjoy daily activities without discomfort. The back pain resulting from a previous injury has become more manageable, enabling me to engage in physical activities with greater ease and freedom.

Physically, Bioelectric Therapy has alleviated almost all of my chronic pain. I have had TMJ since I was 17, and all of my symptoms deriving from that malady are completely gone. My neck muscles are much looser and more flexible. In 2015 I broke my back in 3 places, crushing several vertebrae, leaving me with polyarthritis scoliosis. I have had chronic back pain since. That inflammation is completely gone. The pain that has plagued me since the injury is almost nonexistent. I am able to move freely and am not stiff and hurting every morning.

Furthermore, BioElectric Therapy has played a crucial role in managing my arthritis and the unidentified inflammatory condition. The therapy has helped to reduce inflammation throughout my body, alleviating pain and improving my overall mobility. This newfound relief has allowed me to regain a sense of freedom and independence that I had longed for.

In addition to addressing my physical ailments, BioElectric Therapy has had a profound impact on my emotional and spiritual well-being. The therapy has provided me with invaluable tools for managing anxiety and depression, fostering emotional regulation, and promoting weight management. I have gained a heightened sense of self-awareness, allowing me to better understand and navigate my emotions. This increased self-awareness has been instrumental in my personal growth and has opened doors to profound spiritual experiences.

Mentally and spiritually, I am more able to notice energetic blockages and with the aid of Dan and the BioElectric Therapy, I can shift my energy to clear out those blockages. This therapy has enhanced my self-awareness, which helps me grow spiritually. By clearing out negative or stagnate energy and getting my good energy flowing, I am able to move towards what and whom I am meant to be. It has been an extremely empowering experience.

Bioelectric therapy has treated my anxiety, which for me is a 3-fold issue-mental, physical and spiritual. I have a newfound sense of calm and peace which was not present before I began. With the combination of benefits from the physical, mental and spiritual, I am better able to be who I want to be and focus on what is important to me.

PH! Where do I start? After only my second treatment, my pH was alkaline! I have been trying to get my pH alkaline for years and the treatment did it for me in only 2 sessions. And it has stayed alkaline. Americans don't focus much on pH Level. But those of us who know that our bodies will not function properly in an acidic environment, know how important pH is. Cancer cannot grow in an alkaline environment. Our bodies were made to be alkaline and due to unhealthy food and diets prevalent in our culture, trying to maintain an alkaline body was impossible for me. Now I don't have to do anything. My body pH is where it is supposed to be.

Today, I stand before you as a living testament to the transformative power of BioElectric Therapy. I am no longer defined by anxiety, chronic pain, arthritis, or emotional struggles. Instead, I am filled with a renewed sense of vitality, inner peace, and spiritual growth. BioElectric Therapy has given me the opportunity to reclaim my health, embrace life's challenges with grace, and cultivate a deep connection with my inner self.

To anyone who may be grappling with similar health challenges, I wholeheartedly recommend BioElectric Therapy as a beacon of hope. The compassionate therapists, cutting-edge techniques, and personalized approach provided by this remarkable therapy have the potential to transform your life, just as it has transformed mine. Embrace the opportunity for healing, and let BioElectric Therapy guide you towards a brighter, healthier, and more fulfilling future.
55 Y/O Female with chronic fatigue and pain.

Testimony: At the time I came upon BioElectric Therapy I had undergone two major surgeries; a complete knee replacement and a very serious back surgery to repair pinched nerves and bulging disks. I was both physically and emotionally depleted and did not have enough energy to make it through a day. After the first meeting in which I learned about bioelectrical therapy and what a difference it could make in one's life, I was committed. After 6 months of continuous treatment, I have undergone a change in both my physical health and mental health. My “numbers” have gone from 52% to 78% in the wee time of 6 months. I have more energy and can easily make it through my day. While I did not go in with a major health problem (and I have seen what the therapy can accomplish with those who do have physical challenges), I can tell you that even if you do not feel you need the bioelectrical therapy because, well, you are “just alright as you are”, you still need it! The amount of energy it gives you will have you going through your day with a light step. Mr. Lopez is committed to making this therapy available to as many people as possible, not just the select few. I will continue my therapy and even when my “numbers” reach 80% and beyond, I will feel it necessary to keep going for maintenance. The body is truly an electrical machine.

### Contact Information of Client's Provided Upon Request

### What Does the Data Show?

The data collected from our Health Assessment reports has consistently shown remarkable improvements in clients' well-being. Initially, most individuals scored within the range of 50-55%, indicating a moderate level of health and functioning. However, after undergoing regular monthly BioElectric Therapy treatments, we have observed a significant increase in their scores, with many reaching up to 75-80% within the first 6 month to a year of receiving BioElectric Therapy. This objective data aligns perfectly with the subjective feelings expressed by our clients, who consistently report feeling better, experiencing reduced symptoms, and enjoying an improved quality of life. The correlation between the objective data and the clients' own perceptions further validates the effectiveness of our therapy in providing tangible and measurable benefits for those suffering from chronic conditions.

There are three questions that must be answered for any drug or therapy. Is it safe, is it effective and is it cost effective? The answer to all 3 of these questions is a resounding yes.

### Mission Statement

Our mission is to revolutionize pain management and wellness by integrating Traditional Chinese Medicine (TCM), the Law of Nature, and electrical principles. We believe in a paradigm shift that embraces holistic approaches to healing, focusing on the body's innate ability to restore balance and promote overall well-being.

At the core of our mission is the recognition that pain is not merely a symptom to be masked or numbed, but a signal from the body indicating an underlying imbalance. By drawing upon the ancient wisdom of TCM, we aim to address the root causes of pain and promote long-lasting relief through natural and non-invasive methods.

We also acknowledge the Law of Nature, which emphasizes the interconnectedness of all living beings and the importance of aligning ourselves with the natural rhythms and cycles of the universe. By aligning our treatments with these principles, we strive to restore harmony within the body, mind, and spirit.

Furthermore, we harness the power of electrical principles to enhance the effectiveness of our treatments. By utilizing cutting-edge technology and devices, we aim to optimize the body's electrical pathways, facilitating the flow of energy and promoting healing at a cellular level.

Our mission extends beyond pain management alone. We are committed to empowering individuals to take an active role in their own well-being, providing them with the knowledge and tools to make informed decisions about their health. Through education, guidance, and personalized treatment plans, we aim to empower individuals to achieve optimal wellness and live their lives to the fullest.

### SUMMARY

In summary, our mission is to redefine pain management and wellness by embracing the wisdom of TCM, the Law of Nature, and electrical principles. We strive to provide holistic, natural, and effective solutions that address the root causes of pain, restore balance, and promote overall well-being. Together, let us embark on a journey towards a healthier, pain-free future.

The BioElectric Therapy program non-provisional patent holds tremendous potential to revolutionize the healthcare industry by offering an alternative approach to treating a wide range of chronic conditions. By expanding our reach to a wider audience, we aim to provide relief and improve the quality of life for individuals suffering from ailments such as Inflammation, Autoimmune diseases, Lupus, Epilepsy, Anxiety, Depression, Insomnia, Chronic Fatigue, Sciatica, Ovarian Cyst, Post Traumatic Stress Disorder, and a host of other chronic conditions.

Moreover, our commitment to providing comprehensive care extends beyond the therapy itself. With the inclusion of a Health Assessment report, we offer an objective and measurable way to monitor the progress of the BioElectric Therapy. This report will enable healthcare providers and patients to track improvements, adjust treatment plans, and make informed decisions about their healthcare journey.

By obtaining this non-provisional patent, we not only protect our intellectual property but also pave the way for widespread adoption of this groundbreaking therapy. This will enable us to reach a larger audience, including patients, healthcare providers, and researchers, who can benefit from our approach. Additionally, we are not only securing our position as pioneers in the field but also ensuring that our therapy remains accessible to those who need it most. We firmly believe that everyone deserves the opportunity to explore alternative treatment options and experience relief from their chronic conditions, while having the ability to objectively monitor their progress.

Furthermore, our dedication to expanding our reach aligns with the growing demand for alternative healthcare options. With an increasing number of individuals seeking non-pharmaceutical solutions, our BioElectric Therapy, coupled with the Health Assessment report, offers a comprehensive and personalized approach that can significantly improve their well-being.

In conclusion, the BioElectric Therapy Program non-provisional patent, represents a significant step towards expanding our reach and providing a viable alternative approach to healthcare for individuals suffering from a multitude of chronic conditions. By offering a safe, non-invasive, and drug-free solution, coupled with objective progress monitoring, we aim to improve the lives of countless individuals and contribute to the advancement of healthcare as a whole.

### References

Healing is Voltage—The Handbook-Jerry Tennant, MD MD (H), PSc., D
Earthing—The Most important health discovery ever!-Clinton Ober, Stephen T. Sinatra, M.D. Martin Zucker
PEMF—The 5^{th} Element of Health-Bryant A. Myers
The Body Electric—Electromagnetism and The Foundation of Life-Robert O. Becker, MD Gary Selden
The Electricity for Health in the 21th Century—Carole Punt
The Resonance Effect-How Frequency Specific Microcurrent is Changing Medicine—Carolyn Mc Makin
Book of Chinese Medicine-Timeless Science of Balance and Harmony for Modern Life—Henry H. Sun,
Traditional Chinese Medicine Treatments—Clara Cohen L.AC

1. BioElectric Therapy Center developed a protocol combining electrical and electromagnetic frequencies. This BioElectric Therapy Program has shown promising results in improving the quality of life for individuals living with chronic conditions. This non-invasive and drug-free approach harnesses the power of electrical currents and electromagnetic waves to stimulate the body's natural healing processes, reduce pain, and promote overall well-being. By targeting specific areas of the body, BioElectric Therapy can help alleviate symptoms, improve mobility, and enhance the body's ability to heal itself. With regular monthly sessions, individuals with chronic conditions can experience reduced pain, increased energy levels, improved sleep, and a greater sense of well-being, ultimately leading to a higher quality of life.

2. BioElectric Therapy Center developed a color-coded, easy-to-read Health Assessment report providing a measurable way to monitor the efficacy of BioElectric Therapy. The Health Assessment Report correlates objective measurements with the client's subjective feelings. By utilizing a comprehensive set of quantifiable metrics, our report offers a clear and concise overview of the client's health status after each BioElectric Therapy session. The color-coded system allows for quick identification of improvements or areas that require further attention, providing both the client and therapist with a visual representation of progress. This innovative approach ensures that the client's subjective experiences are validated and supported by objective data, enhancing the overall effectiveness and credibility of this BioElectric Therapy Program as a therapeutic modality.
